(19) United States
(12) Patent Application Publication
(10) Pub. No.: US 2005/0058697 A1
(43) Pub. Date: Mar. 17, 2005

(54) Cell penetrating therapeutic agents

(76) Inventors: Marcel Bally, Bowen Island (CA); Marie-Paul Schultze-Redelmeier, Vancouver (CA); Ghania Chikh, Vancouver (CA)

Correspondence Address: MORRISON & FOERSTER LLP, 3811 VALLEY CENTRE DRIVE, SUITE 500, SAN DIEGO, CA, 92130-2332, US

(21) Appl. No.: 10/727,017

(22) Filed: Dec. 2, 2003

Related U.S. Application Data
(63) Continuation of application No. PCT/CA02/00853, filed on Jun. 7, 2002.
(60) Provisional application No. 60/296,158, filed on Jun. 7, 2001.

(51) Int. Cl. A61K 9/127; C12N 15/88

(52) U.S. Cl. 424/450; 435/458

(57) ABSTRACT

Compositions are provided for delivery of a biologically active agent to a cell, comprising a vehicle having the formula:
A-(BC) wherein: A is a lipid-based vehicle; B is a moiety comprising an internalizing peptide; C is a moiety comprising a biologically active agent; (BC) is a complex comprising B and C in which B is conjugated to C; and, A is conjugated to (BC).

### CROSS-REFERENCE TO RELATED APPLICATIONS

This application claims the benefit of U.S. application No. 60/296,158 filed Jun. 7, 2001, the specification of which is incorporated herein by reference.

### FIELD OF THE INVENTION

The present invention is directed toward intracellular delivery of therapeutic agents.

### BACKGROUND OF THE INVENTION

The efficacy of an agent intended to be used in the treatment of a disease or other condition in a subject is often limited by the pharmacodistribution properties of the agent. Even if a therapeutic agent demonstrates sufficient biological activity in vitro, such activity is not necessarily present after in vivo administration. Firstly, there is the problem of degradation of the agent in the body of the subject while it travels from the site of administration to a desired target cell or tissue. Typically, such an agent will be resident in the bloodstream where it will be subject to degradation. A second problem arises in situations where a therapeutic agent must enter a target cell in order to exert its biological activity. This raises the issue of facilitating entry of the agent into the intracellular environment of a target cell.

The prior art teaches the use of “internalizing peptides” to assist in the transfer of a biologically active agent across cellular membranes. Such internalizing peptides have been referred to in the art as transducing peptides, penetrating, cell membrane penetration agents, and the like. Examples include certain viral and bacterial proteins such as Antennapedia (Antp); the HIV transactivator protein (TAT); the Herpes Virus 22 protein (HSV VP22); and, other peptides containing membrane-translocating amino acid sequences (MTS). Typically, internalizing peptides comprise hydrophobic regions, typically created by the presence of amino acids with amphipathic characteristics. A common characteristic of such internalizing peptides is the ability to carry a hydrophilic moiety, often termed a “cargo molecule” across a biological membrane.

Identification of appropriate amino acid sequences for use in internalizing peptides, construction of internalizing peptides, and their use for carrying cargo molecules across cellular membranes has been well documented in the prior art (for example, see: Rojas, et al. [1998] Nature Biotech. 16:370; Lin, et al. [1995] J. Biol. Chem. 270:14255; Zhang, et al. [1998] P.N.A.S. 95:9184; U.S. Pat. Nos. 5,674,980 and 6,054,312; and, international patent application published under WO 01/15511).

A specific example of the use of an internalizing peptide for intracellular delivery of a biologically active agent (which example will be referred to in more detail below) is found in Schutze-Redelmeier, M. P., et al. (1996) J. Immunol. 157:650. The latter reference describes the preparation of a fusion peptide comprising the homeodomain of Antennapedia (AntpHD) which spontaneously crosses cellular membranes, with an antigenic CTL epitope (Cw3). The aforementioned fusion peptide was capable of crossing the cellular membrane of antigen-presenting dendritic cells (APC) and delivering the epitope into the cytosol of the cells thereby making it available in the MHC class-I antigen presentation pathway. While the Antennapedia peptide was shown to be efficient for causing internalization of the antigen at the target cells, in vivo efficacy could only be achieved when the fusion peptide was administered in the presence of a surfactant adjuvant which protected the fusion peptide until it reached the target.

Lipid-based vehicles such as liposomes have been extensively described in the prior art for delivering biologically active agents to target cells or tissue. Various formulations have been described which are intended to enhance longevity of the vehicle in the bloodstream of a subject, thereby increasing the delivery of agents associated with the vehicle to a target cell or tissue. The usual mechanism by which liposome-encapsulated agents are directed into the cytosol of a target cell is escape of the agent from the endosome, after the liposome is taken into the cell by endocytosis (Zhou, et al. [1994] Immunol. Biology. 190:35).

Internalizing peptides and lipid-based vehicles provide alternate means for intracellular delivery of a biologically active agent. Recently, it was suggested that both systems could be combined in order to protect a complex of an internalizing peptide and a therapeutic agent until delivery of the complex to a target cell (Chikh, G., et al. [2001]. J. Immunological Methods 254:119). In the latter reference, the AntpHD-Cw3 fusion peptide described in Schutze-Redelmeier [supra] was found to spontaneously associate with a liposomal envelope as a result of hydrophobic interaction between the internalizing peptide and lipids on the envelope. The fusion peptide became stably associated with the liposomes and those fusion peptides which were incorporated to the inner lipid envelope were protected against degradation until delivery to APC's. The fusion peptides then disassociated from the liposome and crossed the target cell membrane, thereby passing into the cytosol of the target cell. Transfer of the fusion peptide into the target cell was reported to be independent of interactions between the target cell and the liposome itself. Thus, hydrophobic interaction of the fusion peptide with the liposome permitted the use of a lipid-based vehicle to protect the fusion peptide, without interfering with the ability of the fusion peptide to enter a cell. The weak association of the internalizing peptide with the lipid carrier was sufficient for stability yet still permitted dissociation and transfer of the cargo molecule across the target cell membrane.

### SUMMARY OF THE INVENTION

This invention is based on the discovery that an internalizing peptide/agent complex such as that described above may be conjugated to a lipid-based vehicle without adversely affecting the availability of the complex to cross a cell membrane and enter the cytosol of a target cell. By conjugating the complex to a lipid-based vehicle, greater loading of the complex to the vehicle can be achieved, as well as greater stability during delivery to a target cell than is the case resulting from hydrophobic interaction of the complex with a lipid-based vehicle. Despite the presence of a strong bond between the complex and the vehicle, the complex is still able to enter the cytosol of the target cell at highly efficient rates. Thus, greater efficacy is achieved by conjugating such a complex to a lipid-based vehicle.

This invention provides a composition for delivery of a biologically active agent to a cell, comprising a vehicle having the formula:
A-(BC)
wherein:
A is a lipid-based vehicle;
B is a moiety comprising an internalizing peptide;
C is a moiety comprising a biologically active agent;
(BC) is a complex comprising B and C in which B is conjugated to C; and,
A is conjugated to (BC).

In a composition of this invention, complex (BC) may be conjugated directly to a lipid in the lipid-based vehicle or to any other moiety stably present in the lipid-based vehicle. The conjugation may be by a covalent bond, or may be by means of a linkage comprising a chelated metal. A metal may be chelated in such a linkage through coordination with a chelating group on the lipid-based vehicle and a chelating group present on complex (BC). Such chelating groups may be a metal-chelating moiety such as a metal-chelating lipid or a moiety as typically used as a metal-affinity tag. A common metal-affinity tag is a “his-tag” comprising several histidine residues.

In a composition of this invention, the lipid-based vehicle may be conjugated to complex (BC) through a bond or a linkage directly to B or directly to C, or to another portion of complex (BC) such as a moiety linking B and C or a terminal moiety on B or C.

In a composition of this invention, complex (BC) comprises an internalizing peptide and a biologically active agent conjugated to each other. Thus, a composition of this invention comprises a lipid-based vehicle conjugated to a complex which in turn comprises a biologically active agent intended to be delivered to a cell. The internalizing peptide assists transfer of the biologically active agent across a cell membrane. Although a lipid-based vehicle employed in a composition of this invention may itself comprise a biologically active agent that is the same or different from that found in complex (BC), this invention is directed to delivery of the aforementioned complex.

In a composition of this invention, it is preferable that the linkage or bond between the lipid-based vehicle and complex (BC) be releasable to further facilitate transfer of complex (BC) across a target cell membrane. Preferably, such a releasable bond will disassociate at a pH below normal physiological pH in a mammal in order that the bond or linkage will disassociate when the composition vehicle of this invention reaches a low pH environment such as in the region of a tumor or within the endosome of a cell that has taken up a composition of this invention by endocytosis. Normal physiological pH in a mammal is about 7.4. Thus, a pH releasable bond used in this invention should disassociate at a pH below 7.4, preferably at or below a pH of about 7.0, more preferably at or below a pH of about 6.5 and even more preferably at or below a pH of about 6.0. Thus, in some embodiments of this invention where it is desired to employ a metal-affinity tag for conjugation of the complex to the lipid-based vehicle, it is preferable that the metal-affinity tag has, or comprises moieties having, a pKa of about 6 or more and below that of the normal physiological pH in a mammal. A “his-tag” is particularly suitable for this purpose.

This invention provides injectable pharmaceutical preparations comprising a composition of this invention and a pharmaceutically acceptable carrier.

This invention also provides the use of a composition of this invention for delivery of a biologically active agent to a cell.

This invention also provides the use of a composition of this invention for preparation of a medicament for treatment of a patient by delivery of a biologically active agent to a cell within the patient.

This invention also provides a method of delivering a biologically active agent to a cell in a patient, comprising administering a composition or pharmaceutical preparation of this invention to the patient. Preferably, such administering is by injection.

### BRIEF DESCRIPTION OF THE DRAWINGS

FIG. 1A: is a chart showing organization of the gene used to express AntpHD-Cw3.

FIG. 1B: is a chart showing the amino acid sequence (SEQ ID NO:1) and features of AntpHD-Cw3.

FIG. 2: a graph showing association of AntpHD-Cw3 with DOPC/DOGS-NTA-Ni (10%)/Chol/DSPE-PEG₂₀₀₀ (A) And DOPC/Chol/DSPE-PEG₂₀₀₀ (B) liposomes. Formulations were incubated with AntpHD-Cw3, free peptide was separated from the associated peptide using size exclusion chromatography, amount of peptide (□) and lipid (●) was then determined for each fraction as described herein.

FIG. 3: a graph showing association of AntpHD-Cw3 as a function of initial peptide to liposome ratio. DOPC/Chol liposomes containing 0% (O) and 10% (▪) DOGS-NTA-Ni were incubated with AntpHD-Cw3 at various peptide to liposome ratio for 30 min. at room temperature. The amount of peptide bound was determined after the incubation period as described herein. Data presented represent averaged results obtained from three liposome preparations ±S.E.M.

FIG. 4: a histogram showing the frequency of Cw3-specific T cells as determined by an IFN ELISPOT assay. BALB/c mice were immunized subcutaneously on day 0 with 50 μg AntpHD-Cw3 either in DOPC/Chol/DSPE-PEG₂₀₀₀ or in DOPC/Chol/DSPE-PEG₂₀₀₀/DOGS-NTA-Ni (10%) liposomes. On day 7, spleen cells from individual mice were stimulated for 36 hours with 10 μM Cw3(170-179) and then assayed for the number of Cw3-specific cells as described in Experimental Protocols. Data presented represent averaged results obtained from six mice ±S.E.M.

### DETAILED DESCRIPTION OF THE INVENTION

Abbreviations and Definitions

Throughout this specification, the following abbreviations have the indicated meaning. DOPC: 1,2-Dioleoyl-sn-glycero-3-phosphocholine; DSPC: 1,2-Distearoyl-sn-glycero-3-phosphocholine; NTA: nitrilotriacetic acid; Ni: nickel; DOGS-NTA-Ni: 1,2-Dioleoyl-sn-glycero-3-{[n(5-amino-1-carboxypentyl)iminodiaceticacid]succinyl} (Nickel Salt); CH or Chol: cholesterol; PEG: polyethylene glycol; PEG followed by a number (subscript): the number is the molecular weight of PEG in Daltons; DSPE: 1,2-disteroyl-sn-glycero-3-phosphatidylethanolamine; DSPE-PEG₂₀₀₀:1,2-disteroyl-sn-glycero-3-phosphatidylethanolamine-n-[poly(ethyleneglycol)₂₀₀₀]; AntpHD: Antennapedia homeodomain; AntpHD-Cw3: Antennapedia homeodomain fused to Cw3 epitope; SUV: small unilamellar vesicle; LUV: large unilamellar vesicle; MLV: multilamellar vesicle; CTL, cytotoxic T lymphocyte.

The term “biologically active agent” in this specification means any molecule to be delivered to a cell, including: nucleic acids (which includes oligonucleotides, an entire gene or vector, or any nucleic acid intended to bring about a desired effect in a biological system), peptides (which includes proteins and polypeptides) nucleoproteins, glycoproteins, lipoproteins, polysaccharides and synthetic and biologically engineered analogs thereof; and, drugs and pro-drugs not falling into the foregoing groups. Such agents are active in that they bring about an effect on a biological system, or will bring about such an effect after metabolism or conversion of the agent into an active form. The biological effect may be a therapeutic effect or an ability to modify a physiological response or function. Thus, this term includes “therapeutic agent”. In the context of this invention, a biologically active agent is a “cargo molecule” when associated with an internalizing peptide. This invention is particularly advantageous for use with a biologically active agent that is hydrophilic (having an overall hydrophilic nature) and is unable to cross a cell membrane on its own.

The term “internalizing peptide” in this specification means a peptide sequence that is able to facilitate cellular entry of cargo molecules as described above with respect to the background of the invention. A common attribute of such peptides is their capacity to direct cargo molecules directly into the cytosol following spontaneous crossing of membranes due to the hydrophobicity of the carrier peptide. Possible carrier peptides include, but are not limited to, the TAT protein of HIV TAT, Antennapedia homeodomain (AntpHD), Herpes Virus 22 protein (HSV VP22) as well as non-naturally occurring peptides and modified derivatives of such natural peptides.

A “metal-affinity tag” is a moiety capable of binding (by chelation) to a metal ion and includes short amino acid sequences (generally containing at least two amino acids residues, preferably histidine), typically covalently attached to a moiety to be “tagged”. For example, the tag may be attached to the N- or C-terminus of a peptide. Increasing the number of residues capable of metal binding increases the binding affinity. A peptide modified with a metal-affinity tag can be synthesized by recombinant techniques known in the art followed by isolating and purifying the resulting peptide by methods known in the art. Methods should be employed to ensure that components that interfere with association of the metal-affinity tag to metal (e.g. free histidine or imidazole) are removed. As well, the component modified with the metal-affinity tag can be synthesized synthetically by peptide synthesis.

The terms “metal-chelating moiety” or “metal-chelating group” include any chemical moiety capable of forming a coordinate complex with a metal ion. A “metal-chelating lipid” is a lipid comprising a metal-chelating moiety or group, such as NTA. In the case of a lipid, linkage may be through a lipid head group to the metal-chelating group. A metal having coordination sites free to bond with electron donating elements of the metal-chelating group may then be incorporated. The metal ion may be a transition-metal ion, lanthanide ion, or actinide-metal ion. The metal may be selected from the group consisting of copper, nickel, zinc, iron, cobalt, cadmium, manganese and magnesium. In particular embodiments of this invention, the metal is nickel, cobalt or copper. Copper provides the greatest affinity for a his-tag.

The term “vehicle” as used in this specification means a particle of at least about 50 nanometers diameter which remains suspended in an aqueous solution. The term “vehicle” does not include substances such as excipients that serve only to solubilize a therapeutic agent.

The term “lipid-based vehicle” is a “vehicle” as defined above which comprises one or more lipids. These include liposome, lipid-based vesicles, micelles, lipid-stabilized emulsions, and the like.

The term “liposome” as used herein means vesicles comprised of one or more concentrically ordered lipid bilayers encapsulating an aqueous phase. Formation of such vesicles requires the presence of “vesicle-forming lipids” which are defined herein as amphipathic lipids capable of either forming or being incorporated into a bilayer structure. The latter term includes lipids that are capable of forming a bilayer by themselves or when in combination with another lipid or lipids. An amphipathic lipid is incorporated into a lipid bilayer by having its hydrophobic moiety in contact with the interior, hydrophobic region of the bilayer membrane and its polar head moiety oriented towards an outer, polar surface of the membrane. Most phospholipids belong to the former type of vesicle forming lipid whereas cholesterol is a representative of the latter type. Liposomes can be categorized into multilamellar vesicles, oligolamellar vesicles, unilamellar vesicles and giant liposomes. Multilamellar liposomes (also known as multilamellar vesicles or “MLV”) contain multiple concentric bilayers within each liposome particle, resembling the “layers of an onion”. Unilamellar liposomes enclose a single internal aqueous compartment. Single bilayer (or substantially single bilayer) liposomes include small unilamellar vesicles (SUV) and large unilamellar vesicles (LUV).

In some aspects of this invention, lipid-based vehicles (e.g. liposomes) are prepared to be “cholesterol free”, meaning that such lipid-based vehicles contain “substantially no cholesterol,” or contain “essentially no cholesterol.” The term “cholesterol-free” as used herein with reference to a lipid-based vehicle means that the vehicle is prepared in the absence of cholesterol, or contains substantially no cholesterol, or that the vehicle contains essentially no cholesterol. The term “substantially no cholesterol” allows for the presence of an amount of cholesterol that is insufficient to significantly alter the phase transition characteristics of the lipid-based vesicle (typically less than 20 mol % cholesterol). 20 mol % or more of cholesterol broadens the range of temperatures at which phase transition occurs, with phase transition disappearing at higher cholesterol levels. Preferably, a lipid-based vehicle having substantially no cholesterol will have about 15 or less and more preferably about 10 or less mol % cholesterol. The term “essentially no cholesterol” means about 5 or less mol %, preferably about 2 or less mol % and even more preferably about 1 or less mol % cholesterol. Most preferably, no cholesterol will be present or added when preparing “cholesterol-free” lipid-based vehicles.

In this specification, the terms “conjugate” or “conjugated” refer to moieties connected by one or more bonds between one or more molecules (and thus includes a “linkage”). Such bonds include ionic, covalent, and coordinate bonds, or other such bonds that exist in or by virtue of an aqueous environment and are not affected by the presence of a hydrophobic environment. Thus, the terms exclude hydrophobic association between molecules. Moieties conjugated according to this invention will not disassociate when introduced in or placed in proximity with a hydrophobic environment. Preferred means for conjugation of a lipid-based vehicle to complex (BC) in compositions of this invention are coordinate-covalent linkages or releasable covalent bonds.

Lipid-Based Vehicles

Delivery vehicles are particles that are typically at least about 50 nanometers in diameter, capable of remaining suspended in an aqueous solvent and are able to encapsulate or associate other agents. Preferably the delivery vehicles are 80 to 200 nanometers in diameter. The preferred delivery vehicle is a liposome or lipid-based vesicle. Furthermore, the lipid-based delivery vehicle used in this invention may comprise biologically active agents independent of the agent that is part of the internalizing peptide-agent complex used in this invention. For example, a liposome used in this invention may encapsulate the same or a different biologically active agent as found in the complex or may comprise such an agent as part of or grafted to the liposome envelope.

Liposomes

Liposomes of the present invention or for use in the present invention may be generated by a variety of techniques. These techniques comprise the ether injection method (Deamer et al., Acad. Sci.[1978] 308:250), the surfactant method (Brunner et al., [1976] Biochim. Biophys. Acta, 455:322), the Ca²⁺ fusion method (Paphadjopoulos et al., [1975] Biochim. Biphys. Acta, 394:483), the freeze-thaw method (Pick et al., [1981] Arach. Biochim. Biophys., 212:186) the reverse-phase evaporation method (Szoka et al., [1980] Biochim. Biophys. Acta, 601:559), the ultrasonic treatment method (Huang et al. [1969] Biochemistry, 8:344), the ethanol injection method (Kremer et al. [1977] Biochemistry, 16:3932) the extrusion method (Hope et al., [1985] Biochimica et Biophysica Acta, 812:55) and the French press method (Barenholz et al., [1979] FEBS Lett., 99:210). All of the above processes are basic technologies for the formation of liposome vesicles and these processes can be used in combinations, respectively proved or modified. Preferably, small unilamellar vesicles (SUVs) are prepared by the ultrasonic treatment method, the ethanol injection method and the French press method. Preferably multilamellar vesicles (MLVs) are prepared by the reverse-phase evaporation method or by the simple addition of water to a lipid film followed by dispersal by mechanical agitation (Bangham et al., [1965] J. Mol. Biol. 13:238-252).

A particularly suitable liposome preparation which may be used in the practice of this invention are large unilamellar vesicles (LUVs). LUVs may be prepared by the ether injection method, the surfactant method, the Ca2+ fusion method, the freeze-thaw method, the reverse-phase evaporation method, the French press method or the extrusion method. Preferably, LUVs are prepared according to the extrusion method. The extrusion method involves first combining lipids in chloroform to give a desired molar ratio. A lipid marker may optionally be added to the lipid preparation. The resulting mixture is dried under a stream of nitrogen gas and placed in a vacuum pump until the solvent is substantially removed. The samples are then hydrated in an appropriate buffer or mixture of therapeutic agent or agents. The mixture is then passed through an extrusion apparatus (Northern Lipids, Vancouver, BC) to obtain liposomes of a defined size. Average liposome size can be determined by quasi-elastic light scattering using a NICOMP 370 submicron particle sizer at a wavelength of 632.8 nm. If desired, the resulting liposomes are run down a Sephadex™ G50 column equilibrated with an appropriate buffer in order to remove unencapsulated drug or to create an ion gradient by exchange of the exterior buffer. Subsequent to generation of an ion gradient, LUVs may be encapsulated with therapeutic agents as set forth herein.

Vesicle-forming lipids that may be incorporated into liposomes or lipid carriers of this invention may be selected from a variety of amphipathic lipids, typically including phospholipids such as phosphatidylcholine (PC), phosphatidylethanolamine (PE), phosphatidic acid (PA), phosphatidylinositol (PI), or phosphatidylglycerol (PG); sterols such as cholesterol; sphingolipids such as sphingomyelin and glycolipids. Preferably the vesicle-forming lipids will have two fatty acids, the acyl chains of which being independently selected from the group consisting of caproyl (6:0), heptanoyl (7:0), octanoyl (8:0), nonanoyl (9:0), capryl (10:0), undecanoyl (11:0), lauroyl (12:0), tridecanoyl (13:0), mirystoyl (14:0), pentadecanoyl (15:0), palmitoyl (16:0), heptadecanoyl (17:0), stearoyl (18:0), nonadecanoyl (19:0), arachidoyl (20:0), heniecosanoyl (21:0), behenoyl (22:0), tricosanoyl (23:0), lingnoceroyl (24:0), cerotoyl (26:0) and phytanoyl, including the unsaturated versions of these fatty acid chains in the cis or trans configurations such as oleoyl (18:1), linoleoyl (18:2), erucoyl (20:4) and docosahexaenoyl (22:6).

Mixtures of lipids may also be used for the purpose of this invention. Preferably, lipids are selected that differ by less than four methylene groups in the fatty acid chain. Lipid mixtures used in the examples described herein were: DOPC/Chol, DOPC/Chol/DSPE-PEG₂₀₀₀, DOPC/DOGS-NTA-Ni/Chol, DOPC/DOGS-NTA-Ni/Chol/DSPE-PEG₂₀₀₀, DSPC/Chol, DSPC/DOGS-NTA-Ni/Chol and DSPC/DOGS-NTA-Ni/Chol/DSPE-PEG₂₀₀₀ at various mol % ratios.

Liposomes for use in this invention may comprise substantially no cholesterol, essentially no cholesterol or no cholesterol. The incorporation of less than 20 mol % cholesterol in liposomes can allow for retention of drugs not optimally retained when liposomes are prepared with greater than 20 mol % cholesterol. Additionally, liposomes prepared with less than 20 mol % cholesterol display narrow phase transition temperatures, a property that may be exploited for the preparation of liposomes that release encapsulated agents due to the application of heat (thermosensitive liposomes).

Therapeutic lipids may be incorporated into liposomes or lipid carriers of this invention. Possible candidate therapeutic lipids are selected from ether lipids, phosphatidic acid, phosphonates, ceramide, dihydroxyceramide, phytoceramide, sphingosine, sphingomyelin, serine-containing lipids, phosphatidylserine and sphinganine.

A therapeutic lipid can be incorporated within the lipid film such that when the delivery vehicle is generated, the therapeutic lipid is incorporated within the bilayer of the delivery vehicle. By way of example, if the lipid film containing the therapeutic lipid is hydrated with a therapeutic agent, the drug will be encapsulated in the delivery vehicle containing a therapeutic lipid in the bilayer. For example, a lipid film containing a therapeutic lipid can be hydrated with a solution comprising a therapeutic agent to produce a formulation comprising a therapeutic lipid and a therapeutic agent encapsulated in the aqueous interior of the liposome. Alternatively, the therapeutic lipid may be exchanged into the liposomes after they are generated with an encapsulated agent. The therapeutic lipid may be soluble in aqueous buffer or aided with the use of detergents or ethanol. Incubation of the therapeutic lipid solution with preformed liposomes may allow for lipid transfer into the liposomal bilayer. The liposomes can subsequently be purified though column chromatography or dialysis to remove any unincorporated lipid.

Enhanced targeting to specific cell populations may be achieved by linking targeting ligands (such as sugar moieties, cell receptor ligands, and the like) to the surface of a liposome to optimize binding to target cells.

Liposomes for use in this invention may be from 50 nm to about 1 μm in diameter. However, preferred liposomes of this invention will be less than about 200 nm, preferably less than about 160 nm, and more preferably less than about 140 nm in diameter. 100-140 nm liposomes are employed in the examples below. Liposomes are typically sized by extrusion through a filter (e.g. a polycarbonate filter) having pores or passages of the desired diameter.

Preferably, liposomes will comprise at least 80 mol % of a phospholipid having two fatty acids, the acyl chains of each having at least 18 carbon atoms. A preferred phospholipid with acyl chains of 18 carbon atoms is DOPC. Also preferred is that the liposomes comprise at least 5 mol % of a lipid that is capable of forming a releasable chemically covalent bond. Also preferred are liposomes comprising at least 5 mol % of a metal-chelated lipid.

Liposomes of this invention may comprise a hydrophilic moiety, including but not limited to phosphatidylglycerol, phosphatidylinosol, or a polyalkylether such as PEG. Grafting a hydrophilic moiety to the surface of liposomes can “sterically stabilize” liposomes thereby maximizing the circulation lifetime of the carrier. This results in enhanced blood stability and increased circulation time, reduced uptake into healthy tissues, and increased delivery to disease sites such as solid tumors (see U.S. Pat. Nos. 5,013,556 and 5,593,622; and Patel et al., [1992] Crit Rev Ther Drug Carrier Syst, 9:39). Typically, the hydrophilic moiety is conjugated to a lipid component of the liposome. We have found that none or minimal liposome-liposome cross linking occurs when short his-tags are used in this invention. Increasing the number of histidine residues in a his-tag to about ten results in aggregation in some cases. Therefore, the present invention may be adapted to use liposomes without incorporation of hydrophilic moieties in the liposome.

Micelles

Micelles for use in this invention may be produced from amphipathic lipids or lipoproteins and may also comprise non-lipid polymers. Preferred micelles are less than 500 nm in diameter, most preferably less than 200 nm.

Micelles used in this invention may be produced from combinations of amphiphilic lipid materials well known and routinely utilized in the art to produce micelles. At least one lipid component may be covalently bonded to a water-soluble polymer. Lipids may include relatively rigid varieties, such as sphingomyelin, or fluid types, such as phospholipids having unsaturated acyl chains. The lipid materials may be selected by those of skill in the art in order that the circulation time of the micelles be balanced with the drug release rate. Preferably, leakage of the drug from the micelle is significantly less than the plasma distribution rate. Polymers covalently bonded to the lipid component may include any compounds known and routinely utilized in the art of sterically stabilized liposome technology and technologies which are useful for increasing circulatory half-life for proteins, including for example polyvinyl alcohol, polylactic acid, polyglycolic acid, polyvinylpyrrolidone, polyacrylamide, polyglycerol, polyaxozlines, or synthetic lipids with polymeric head groups. Preferred lipids for producing micelles according to the invention include distearoyl-phosphatidylethanolamine covalently bonded to a hydrophilic-lipid conjugate alone or in further combination with phosphatidylcholine (PC).

Micelles may be prepared from lipoproteins or artificial lipoproteins including low density lipoproteins, chylomicrons and high density lipoproteins. Artificial lipoproteins may also comprise lipidized protein with targeting capabilities. Uptake of lipoproteins into cell populations may be facilitated by receptors present on the target cells. For instance, uptake of low density lipoproteins into cancerous cells may be facilitated by LDL receptors present on such cells and uptake of chylomicrons and lactosylated high density lipoproteins into hepatocytes may be facilitated by the remnant receptor and the lactosylated receptor respectively.

Lipid Stabilized Emulsions

Micelles may be prepared such that they comprise an oil filled core stabilized by an emulsifying component such as a monolayer or bilayer or lipids. The core may comprise fatty acid esters such as triacylglycerol (corn oil). The monolayer or bilayer may comprise a hydrophilic polymer lipid conjugate. These vehicles may be prepared by homogenization of the oil in the presence of liposomes. Therapeutic agents that are poorly water soluble are typically incorporated into such emulsions.

Internalizing Peptide/Biologically Active Agent Complexes

This complex, identified by the symbol (BC) above, comprises an internalizing peptide conjugated to the biologically active agent intended to be delivered to a cell and for which the internalizing peptide will assist in transfer across a cell membrane. The complex may comprise additional components which may be transferred across a cell membrane with the internalizing peptide or which may be intended to be released or cleaved before administration, during transit in the patient, or at the site of a target cell or tissue.

Means for conjugating a biologically active agent to a peptide may be any such means known in the art and will depend upon the nature of the agent to be conjugated, the make-up of the peptide, and the preferred means of production of the complex. Covalent attachment of a variety of compounds, including peptides, nucleic acids, non-peptide/nucleic acid drugs and the like to a peptide are well known in the art. For example, attachment of cargo molecules to internalizing peptides is described in WO 01/15511. One typical method involves formation of an amide bond between a carboxylic acid on an agent with an amino group on a peptide. Recombinant techniques may be employed if the agent is itself a peptide since the agent and the internalizing peptide could be expressed as a fusion peptide.

Conjugation of Complex to Lipid-Based Vehicle

Methods for conjugation of peptides and non-peptide moieties to lipid-based carriers are well known and the method of choice will depend upon the nature of the complex to be conjugated and the nature of the component in the vehicle to which the complex is to be conjugated. Conventional methods include conjugating peptides to lipids through covalent cross-linking of the peptide to a phospholipid. A typical method involves formation of an amide bond between a carboxylic acid on a lipid and an amino group on a peptide. Examples include the conventional methodologies for conjugation of hydrophilic polymers to lipids in a liposomal envelope or attachment of non-peptide moieties to a lipid in a lipid-based vehicle.

Complex (BC) is not necessarily conjugated directly to a lipid in the lipid-based vehicle. Instead, the complex may be conjugated to a non-lipid moiety which itself is conjugated to a lipid or is otherwise stably present in the lipid-based vehicle.

Preferably, conjugation of complex (BC) to a lipid-based vehicle in this invention will be by means of a releasable bond or linkage. Releasable bonds or linkages are typically reducible chemical linkages, such as disulfide, ester or peptide linkages, light sensitive linkages, heat sensitive linkages or pH sensitive linkages. The aforementioned may be covalent bonds that can be released by a suitable cleaving ‘agent’, such as a reducing agent, a hydrolytic enzyme, a photolytic stimulus or a change in temperature or pH. Examples of such linkages that may be used in this invention for conjugation of a lipid to the complex of an internalizing peptide and agent are described in the international patent applications published under WO 89/11867 and WO 91/12023.

pH sensitive linkages take advantage of the fact that a molecule can have an affinity for a second molecule at one pH yet release that molecule in response to a change in pH. The bonding and release may be due to a number of reasons. In one example, a reduced or elevated pH can cause a molecule to undergo a conformational change which may alter the binding affinity of that molecule. Another example may be due to breakage of an ionic bond or a coordinate bond due to the altered pH. This phenomenon is commonly encountered when working with interactions between a metal and various metal-affinity tags. Exploiting the high affinity between a metal-affinity tag and metal(s) is the principle behind one type of affinity chromatography. Linking molecules with a metal-affinity tag to a lipid-based delivery vehicle containing metal-chelating lipids is one method of conjugating complex (BC) to a lipid-based vehicle.

Van Broekhoven and Altin described recombinant cell receptors, each possessing a metal-affinity tag, ‘engrafted’ onto fluorescently labeled liposomes through a metal-chelating linkage for the detection of low-affinity receptor-ligand interactions (Immunol. Cell Biol. 79:274 [2001]). Similarly, described in international patent applications published under WO 00/64471 and WO 01/28569 and in U.S. Pat. No. 6,087,452, targeting ligands conjugated with a metal-affinity tag are attached to delivery vehicles through metal-chelation to enhance the targeting of complexes to a particular cell or tissue of interest. These conjugation methods exploit the interaction between transition metals and metal-affinity tags for the purpose of stably attaching targeting ligands used to direct the delivery of liposome encapsulated agents to a desired cell type(s) and are particularly suitable for use in this invention for conjugating a lipid to the complex.

Conjugation of complex (BC) to a lipid-based vehicle in this invention through metal-chelation may be carried out according to any of the methods described in the literature referenced above. A particularly convenient methodology employs the use of metal-chelating lipids in the lipid-based vehicle and a metal-affinity tag on complex (BC). Various metal-chelating lipids are commercially available such as the ones described in the Examples below. A useful metal-affinity tag which is particularly suitable for conjugation to peptides is a tract of amino acids having side chains capable of forming a coordinate complex or chelating with a metal ion. A common example is a “his-tag” which will consist of several adjacent histidine residues. A his-tag is a particularly suitable moiety for use in conjugating complex (BC) to a lipid-based vehicle in this invention since such a complex will disassociate at a pH below that of normal physiological pH. This facilitates release of the complex from the lipid-based vehicle in the more acidic environment of an endosome or in the region of some tumor cells. Immediate release of the complex in the acidic environment of an endosome will enhance delivery of the complex into the cytosol of the target cell.

Metal-chelating moieties present in a lipid-based vehicle for use in this invention may be any of a number of known moieties capable of forming a coordinate complex with metal ions and thereby capable of participating in a chelation linkage with complex (BC). A common chelating moiety which is employed in commercially available metal-chelating lipids is NTA. However, other compounds that might be incorporated in a lipid-based vehicle may be employed for chelation of a metal. Metal coordination sites may exist or be identified as being present in a variety of compounds comprising appropriate arrangement of hydroxyl, carbonyl, amide or amine substituents. For example, the compounds daunorubicin and doxorubicin have well positioned hydroxyl side groups capable of forming complexes with copper or iron. The latter compounds are typically incorporated in lipid-based vehicles and may be exploited as metal-chelating moieties for use in this invention.

Therapeutic Uses

Compositions of this invention may be used for delivery of agents for treatment of a variety of diseases in warm-blooded animals or for bringing about a desired biological effect such as an immune response. Examples of medical uses of the compositions of the present invention include treating cancer, treating cardiovascular diseases such as hypertension, cardiac arrhythmia and restenosis, treating bacterial, viral, fungal or parasitic infections, treating and/or preventing diseases through the use of the compositions of the present inventions as vaccines, treating inflammation or treating autoimmune diseases.

Tumor vasculature is generally leakier than normal vasculature due to fenestrations or gaps in the endothelia. This allows the delivery vehicles of 200 nm in diameter or less to penetrate the discontinuous endothelial cell layer and underlying basement membrane surrounding the vessels supplying blood to a tumor. Selective accumulation of the delivery vehicles into tumor sites following extravasation leads to enhanced delivery and effectiveness of the therapeutic agent. In order to promote extravasation, targeting ligands against tumor associated endothelial cells may be bound to the outer surface of the delivery vehicles. Furthermore, tumors are naturally acidic and the low pH environment is ideal for the increased release of agent both in and around the tumor site when a pH sensitive linkage is employed in the composition of this invention.

Administering Lipid-Based Vehicles

Compositions of the present invention may be administered to warm-blooded animals, including humans. For treatment of human ailments, a qualified physician will determine how the compositions of the present invention should be utilized with respect to dose, schedule and route of administration using established protocols. Such applications may also utilize dose escalation should therapeutic agents associated with hybrid delivery system compositions of the present invention exhibit reduced toxicity to healthy tissues of the subject.

Preferably, the compositions of the present invention are administered parenterally, i.e., intraarterialy, intravenously, intraperitoneally, subcutaneously, or intramuscularly. More preferably, the pharmaceutical compositions are administered intravenously or intraperitoneally by a bolus injection. For example, see Rahman et al., U.S. Pat. No. 3,993,754; Sears, U.S. Pat. No. 4,145,410; Papahadjopoulos et al., U.S. Pat. No. 4,235,871; Schneider, U.S. Pat. No. 4,224,179; Lenk et al., U.S. Pat. No. 4,522,803; and

Fountain et al., U.S. Pat. No. 4,588,578.

In other methods, compositions of the present invention can be contacted with the target tissue by direct application of the preparation to the tissue. The application may be made by topical, “open” or “closed” procedures. By “topical”, it is meant the direct application of the pharmaceutical preparation to a tissue exposed to the environment, such as the skin, oropharynx, external auditory canal, and the like. “Open” procedures are those procedures include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrazamide imaging of the spinal cord. Alternatively, the preparations may be administered through endoscopic devices.

Pharmaceutical preparations containing compositions of this invention may be prepared according to standard techniques and may comprise water, buffered water, 0.4% saline, 0.3% glycine, 5% dextrose and the like, including glycoproteins for enhanced stability, such as albumin, lipoprotein, globulin, and the like. These preparations may be sterilized by conventional, well-known sterilization techniques. The resulting aqueous solutions may be packaged for use or filtered under aseptic conditions and lyophilized, the lyophilized preparation being combined with a sterile aqueous solution prior to administration. The preparations may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. Additionally, the preparation may include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

The concentration of compositions of this invention in a pharmaceutical formulation can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment. Alternatively, preparations composed of irritating lipids may be diluted to low concentrations to lessen inflammation at the site of administration. For diagnosis, the amount of composition administered will depend upon the particular label used, the disease state being diagnosed and the judgment of the clinician.

Preferably, pharmaceutical preparations and compositions of the present invention are administered intravenously. Dosage for the hybrid system formulations will depend on the ratio of drug to lipid and the administrating physician's opinion based on age, weight, and condition of the patient.

The methods of the present invention may be practiced in a variety of hosts. Preferred hosts include mammalian species, such as humans, non-human primates, dogs, cats, cattle, horses, sheep, and the like.

### EXAMPLES

AntpHD-Cw3 Construction

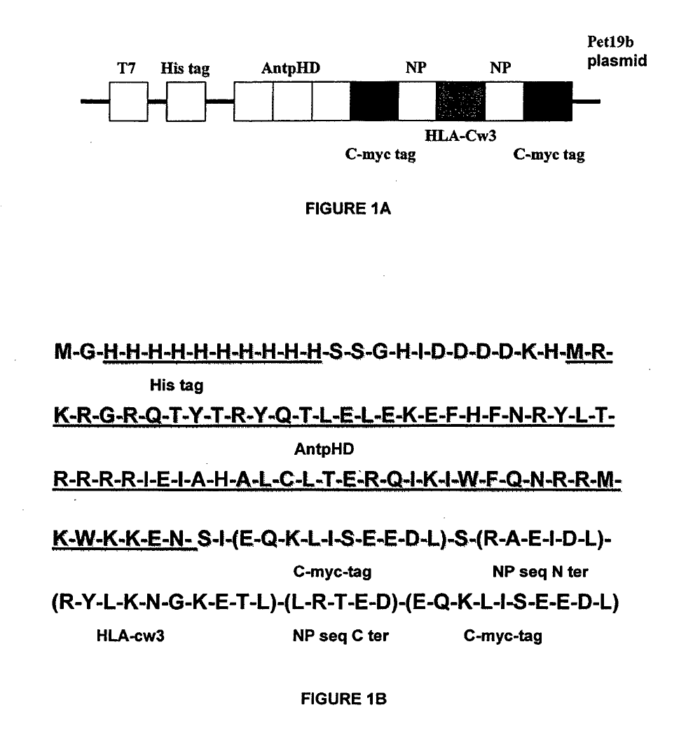

AntpHD-Cw3 peptide cloned in pAH61S plasmid (Schutze-Redelmeier et al., [1996] J Immunol. July 15;157(2):650) was sub-cloned into pET19 (Novagen, Inc., Madison, Wis.) between NdeI and BamH1 under the control of a T7 promoter. The peptide was expressed as a fusion peptide containing 10 histidine residues (his-tag) and a 13-amino acid linker attached the amino terminus of a 61 amino acid tract (AntpH) containing the Antp homeodomain, as well as a 10 amino acid tract corresponding to amino acids 170-179 of Cw3 (FIG. 1). For peptide expression, Escherichia coli strain 15, BL21 (DE3)Lys was used, it contains the T7 RNA polymerase gene under control of lac promoter (Studier et al., [1990] Methods Enzymol. 185:60). Peptide was purified as described in Chikh et al., [supra] by nickel-chelate affinity resin according to manufacturer's instructions (Qiagen, Chatsworth, Calif.). The eluted fractions were analyzed by SDS-PAGE on 15% gels and Coomassie blue staining. Purity was assessed at 80-90%. Before incubation with liposomes the peptide was dialyzed against 50 mM NaH₂PO₄, 400 mM NaCl, pH8 buffer in order to remove the imidazole, which might interfere with the peptide association with DOPC/DOG-NTA-Ni/CHOL/DSPE-PEG 2000 liposomes.

AntpHD-Conjugated Liposomes

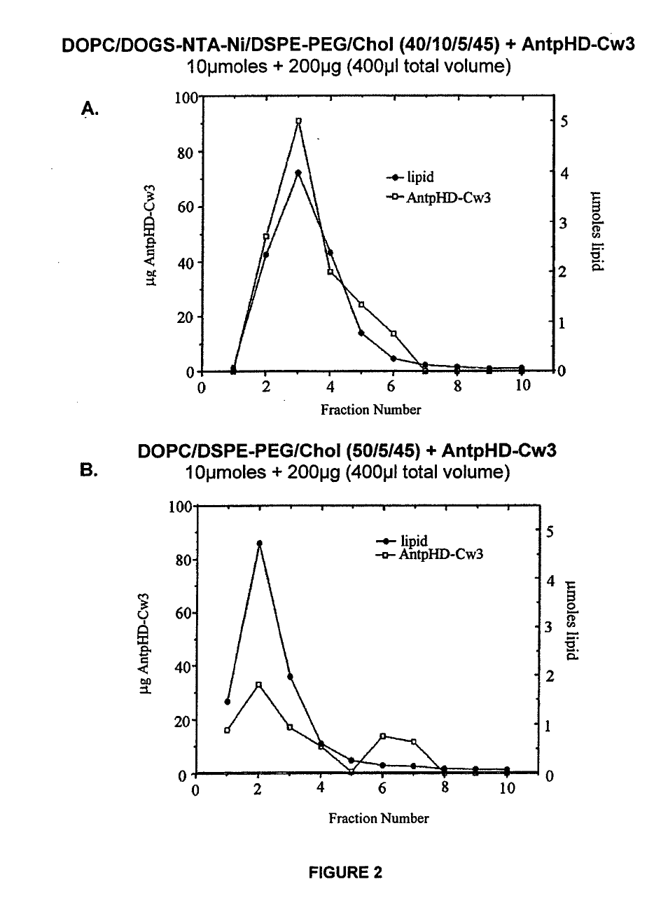

200 μg of dialyzed AntpHD-Cw3 was incubated with 10 μmoles liposomes as described for FIGS. 2-4 (v/v) for 30 min. at room temperature under rotary shaking. The mixture was then loaded on a Biogel™ A 1.5 m chromatography column in order to remove the free peptide from peptide that was incorporated into the liposome. The peptide/liposome ratio was determined by quantifying liposomal lipid using [³H]CHE. Radioactivity was measured by liquid scintillation counting on a Can berra-Packard scintillation P counter (1900 TR Tri Carb™), using Pico-fluor 40™ scintillation cocktail. The peptide was quantified using the micro BCA™ protein assay kit, after dissolving the liposomes in 0.5% triton. BSA was used as the standard for this analysis.

Association of Antennapedia with Liposomes Using Metal-Affinity Tag

In the prior art, AntpHD-Cw3 was used to deliver the antigenic peptide Cw3 to the major MHC class I presentation pathway and to prime cytotoxic T cells (CTL). However, in vivo use of the recombinant peptide was limited because CTLs could only be primed in the presence of sodium dodecyl sulfate (SDS), a toxic adjuvant for humans. However, hydrophobic association with liposomes protected the AntpHD-Cw3 peptides from serum degradation in the absence of SDS and delivery of the recombinant peptide. AntpHD-Cw3 into the MHC class I pathway of antigen presenting cells was increased (Chikh, G., et al. [supra]).

In the present example, a high affinity bond was tested between histidine-tagged AntpHD-Cw3 and nickel chelated to a lipid (DOGS. NTA Ni) in the liposome. Results using liposomes comprising DOPC/DOGS. NTA Ni/DSPE-PEG₂₀₀₀/chol showed 100% encapsulation efficiency (FIG. 2A) whereas the same formulation without the metal-chelated lipid gave 40% efficiency (FIG. 2B).

Binding Efficiency

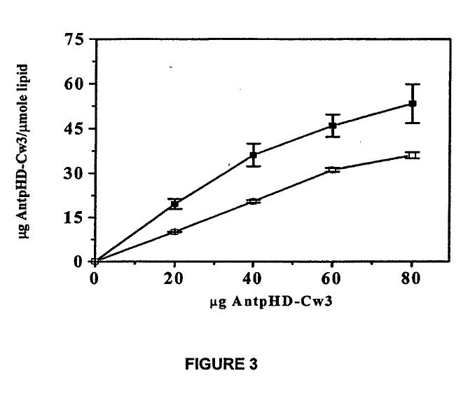

10 mol % DOGS-NTA-Ni provided optimal AntpHD-Cw3 binding attributes (peptide binding in the absence of aggregation) and using this concentration, the influence of initial AntpHD-Cw3 to peptide to lipid ratio on binding efficiency was characterized. The results shown in FIG. 3 demonstrate that saturation occurred around 50 μg AntpHD-Cw3/μmole liposomal lipid. Increasing the initial ratio to 60 and 80 AntpHD-Cw3/μmole lipid caused a decrease in the coupling efficiency to 80% and 65%, respectively. Consequently, mice were immunized subcutaneously on day 0 with 50 μg AntpHD-Cw3/μmole lipid.

Characterization of the Binding Specificity

The presence of 166 mM imidazole was sufficient to inhibit the binding of histidine-tagged AntpHD-Cw3 to nickel-chelated liposomes. This “competitive inhibitor” reaction demonstrated that liposomal uptake of the recombinant peptide in the presence of DOGS-NTA-Ni lipids occurred as a result of binding through a metal-affinity tag to metal coordinate covalent linkage, as opposed to incorporation occurring as consequence of hydrophobic interaction.

Immunization of Mice

Groups of BALB/c mice (6 per group), 6 to 8 weeks of age, were immunized s.c. once on day 0. On day 7, spleens were harvested and immune response was monitored using an Elispot assay.

Elispot Assay

The ELISPOT assay described by Murali-Krishna et al. was modified to detect Cw3-specific CD8+ cells (Immunity 8:177 [1998]). ELISPOT plates (Multiscreen-IP Clear™ plates, Millipore) were coated overnight at 4° C. with capture anti-INFγ antibody (2 μg/ml) (clone R4-6A2) (Pharmingen). The plates were then blocked with 1% BSA in PBS for 2h at room temperature. After 3 washes, responder cells in RPMI medium supplemented with 10% ConA supernatant containing TCGF (T cell growth factors), 10% FCS, 1% glutamine, 1% penicillin/streptomycin and 5.10⁻⁵ M P-2-ME were added to the wells along with 5×10⁵ irradiated syngeneic feeder cells. Cells were incubated for 36h in the presence or absence of 10 μM of Cw3(170-179) peptide. After culture, the plates were washed and biotinylated anti-INFγ detection antibody (clone XMG1.2) (Pharmingen) was added (1 μg/ml) and the plates were then incubated for 1 h at room temperature. Spots developed following addition of freshly prepared HRP diluted 1:2000 in PBS/Tween containing 1% BSA, followed by repeated (5×) washes with PBS/Tween and addition of 200%1 of HRP substrate (Opti-4CN™ substrate kit, Bio-Rad, Hercules, Calif.). The frequency of peptide-specific T cells was calculated based on the percentage of cells present in the responding population.

Assessment of Biological Activity

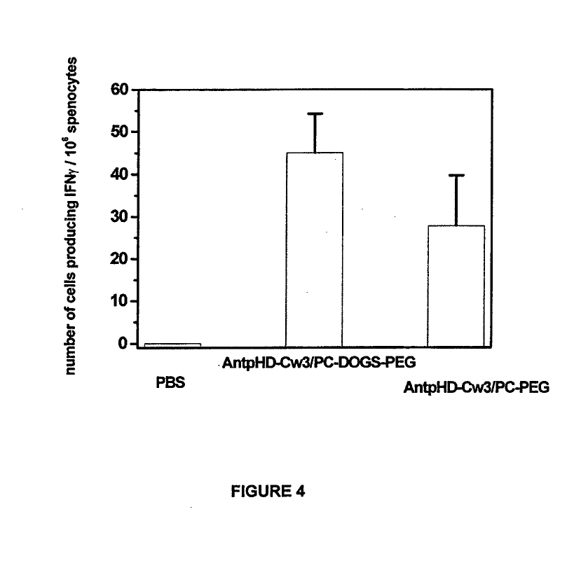

Results from the elispot assays monitoring immune system stimulation by measuring the frequency of splenocytes secreting IFNγ, showed that a single immunization with 50 μg AntpHD-Cw3 contained on the DOGS-NTA-Ni liposomes was able to stimulate the immune system specifically against Cw3 peptide. Importantly, the results illustrated in FIG. 4 show that the frequency of splenocytes secreting IFNγ, obtained with injection of the peptide in the composition of this invention was much higher than the frequency obtained using the formulation prepared through spontaneous association of the peptide with a liposome through hydrophobic interaction. Pharmacokinetic analysis of the DOGS-NTA-Ni indicated that there was little in the way of a detectable immune response to this lipid alone.

Although the foregoing invention has been described in some detail by way of illustration and example for the purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

1. A composition for delivery of a biologically active agent to a cell, the composition comprising lipid-based vehicles which comprise conjugates of the formula:
A-(BC)
wherein:
A is a component of said vehicles;
B is a moiety comprising an internalizing peptide;
C is a moiety comprising a biologically active agent;
(BC) is a complex comprising B and C in which B is conjugated to C; and,
A is conjugated to (BC).

2. The composition of claim 1, wherein (BC) is conjugated to a lipid in A.

3. The composition of claim 1 or 2, wherein A is conjugated to (BC) by a coordinate covalent linkage.

4. The composition of claim 3, wherein the linkage is between a metal-chelating moiety and a metal-affinity tag, wherein the chelating moiety and affinity tag form a complex with a metal ion.

5. The composition of claim 4, wherein the affinity tag is on (BC).

6. The composition of claim 5, wherein the metal-chelating moiety is a metal-chelating lipid in A.

7. The composition of any one of claims 4-6, wherein the metal-affinity tag has a pKa of about 6 or more.

8. The composition of any one of claims 4-7, wherein the metal-affinity tag disassociates from the metal-chelating moiety at a pH below normal physiological pH in a mammal.

9. The composition of claim 8, wherein the tag begins to disassociate at or below about pH 6.5.

10. The composition of any one of claims 4-9, wherein the metal-affinity tag is a tract of two or more amino acids having a pKa of about 6 or more.

11. The composition of any one of claims 4-10, wherein the metal-affinity tag is a his-tag.

12. The composition of any one of claims 4-11, wherein the metal ion is an ion of a metal selected from the group consisting of copper, nickel, zinc, iron, cobalt, manganese and magnesium.

13. The composition of claim 12, wherein the metal is nickel.

14. The composition of claim 12, wherein the metal is copper.

15. The composition of claim 12, wherein the metal is cobalt.

16. The composition of any one of claims 4-15, wherein the metal-chelating moiety comprises NTA.

17. The composition of claim 1 or 2, wherein A is conjugated to (BC) by a covalent bond.

18. The composition of claim 17, wherein the covalent bond is a releasable bond.

19. The composition of claim 18, wherein the releasable bond dissociates at a pH below normal physiological pH in a mammal.

20. The composition of claim 18, wherein the releasable bond begins to dissociate at or below about pH 6.5.

21. The composition of any one of claims 1-20, wherein A is conjugated to B.

22. The composition of any one of claims 1-20, wherein A is conjugated to C.

23. The composition of any one of claims 1-22, wherein the internalizing peptide of B is derived from Antennapedia.

24. The composition of any one of claims 1-23, wherein the biologically active agent of C is hydrophilic.

25. The composition of any one of claims 1-24, wherein the biologically active agent of C is selected from the group consisting of a peptide, a nucleic acid, and a drug that is not a peptide or nucleic acid.

26. The composition of claim 25, wherein C is a peptide and (BC) is a fusion peptide.

27. The composition of claim 26, wherein (BC) is a recombinant peptide.

28. A composition according to any one of claims 1-27, wherein said vehicles comprise a biologically active agent.

29. The composition of any one of claims 1-28, wherein said vehicles are liposomes.

30. The composition of any one of claims 1-29, wherein the biologically active agent of C is an antigen for eliciting an immune response.

31. An injectable pharmaceutical preparation comprising a composition according to any one of claims 1-30, and a pharmaceutically acceptable carrier.

32. The use of a composition according to any one of claims 1-30, for delivery of C to a cell.

33. The use of a composition according to any one of claims 1-30, for preparation of a medicament for treatment of a patient by delivery of C to a cell within the patient.

34. A method of delivering a biologically active agent to a cell in a patient, comprising administering a composition according to any one of claims 1-30, to the patient.

35. The method of claim 34, wherein the administering is by injection.
